(19) United States
(12) Patent Application Publication
(10) Pub. No.: US 2025/0114165 A1
(43) Pub. Date: Apr. 10, 2025

(54) FIDUCIAL MARKER

(71) Applicant: AUGMEDICS LTD., Yokneam Illit (IL)

(72) Inventors: Stuart Wolf, Yokneam (IL); Nissan Elimelech, Beerotaim (IL)

(21) Appl. No.: 18/949,164

(22) Filed: Nov. 15, 2024

Related U.S. Application Data
(63) Continuation of application No. 17/585,629, filed on Jan. 27, 2022, now Pat. No. 12,178,666.
Application No. 17/585,629 is a continuation-in-part of application No. PCT/IB2020/056893, filed on Jul. 22, 2020.
Application No. PCT/IB2020/056893 is a continuation-in-part of application No. 16/524,258, filed on Jul. 29, 2019, now Pat. No. 11,980,506.

(51) Int. Cl. A61B 90/00; A61B 90/57; G02B 27/01

(52) U.S. Cl. CPC A61B 90/39; A61B 90/361; A61B 90/57; G02B 27/017; A61B 2090/365; A61B 2090/3966; A61B 2090/3983

(57) ABSTRACT

A medical marking device, consisting of a radiotransparent plate having a first plurality of radiopaque elements embedded therein in a first predefined pattern and a second plurality of optical reflectors positioned on a surface in proximity to the plate in a second predefined pattern. The device also includes a sigmoid mounting arm having a first end connected to the radiotransparent plate and a second end containing one or more fastening receptacles.

### CROSS-REFERENCE TO RELATED APPLICATIONS

This application is a continuation of U.S. patent application Ser. No. 17/585,629, filed Jan. 27, 2022, which is a continuation in part of PCT Patent Application PCT/IB2020/056893, filed Jul. 22, 2020, which is a continuation in part of U.S. patent application Ser. No. 16/524,258, filed Jul. 29, 2019. Each of the foregoing applications is hereby incorporated by reference herein in its entirety.

### FIELD OF THE INVENTION

The present invention relates generally to a fiducial marker, and specifically to a marker that can be used for registration of multiple frames of reference present in image guided surgery.

### BACKGROUND OF THE INVENTION

In an augmented reality system that is used for image guided surgery the system should track objects used in the surgery, and/or elements of the patient undergoing the surgery. The tracking requires registration of different frames of reference operative during the surgery, including a frame of reference of the patient and a frame of reference of a fluoroscopy facility imaging the patient. The registration typically requires a fiducial marker, and a number of such markers are known. References describing examples of markers are given below.

U.S. Pat. No. 6,314,310 to Ben-Haim et al. describes apparatus for X-ray guided surgery. The apparatus includes a reference element which is placed in contact with the body of a subject, and the element includes a plurality of fiducial marks.

U.S. Pat. No. 7,107,091 to Jutras et al. describes a surgical device that is adapted for use with an image guided surgical system. The device facilitates monitoring inter-dependently mobile bone elements.

U.S. Pat. No. 9,179,984 to Teichman et al. describes a navigation system that includes a multi-configuration tracking array. A plurality of tracking devices can be positioned on the multi-configuration tracking array.

U.S. Pat. No. 9,498,231 to Haider et al. describes computer aided surgery utilizing an on tool tracking system.

U.S. Pat. No. 9,844,413 to Doan et al. describes a monitoring system that tracks the non-visible structure of a body in three dimensions. A tracker obtains image information of an object and instruments in its vicinity, all bearing 3D tracking markers with at least one pattern segment.

U.S. Pat. No. 9,872,733 to Shoham et al. describes a system providing a mechanical guide for drilling the holes for distal screws in intramedullary nailing surgery. The drill guide is automatically positioned by a robot relative to the distal locking nail holes, using data derived from X-ray fluoroscopic images.

U.S. Pat. No. 10,034,713 and U.S. Patent Application 2017/0252109 to Yang et al. describe a system for tracking a position and orientation of a handheld implement. A support member secures one or more markers relative to a longitudinal portion of the handheld implement, and a marker plane containing the markers is orientated at an angle relative to a longitudinal axis of the longitudinal portion.

U.S. Pat. No. 10,080,616 to Wilkinson et al. describes a system that accesses image data of a bone to which a reference marker array is fixed.

U.S. Pat. No. 10,085,709 and U.S. Patent Application 2017/0164919 to Lavallee et al. describe projecting a 3D image on at least part of 2D X-ray images and adjusting projective geometry data of the images, the adjustment comprising registration of the images with the projection of an initial 3D image using an image-to-image registration technique.

U.S. Pat. No. 10,166,079 to McLachlin et al. describes performing intraoperative image registration during a medical procedure. A depth-encoded marker is provided to an object of interest. The marker is imageable by at least two imaging systems, and the marker has asymmetry in at least a depth dimension.

U.S. Pat. No. 10,194,993 to Roger et al. describes a system for aiding surgery on a patient. The system includes a display device and a storage device that stores an image of at least a portion of the anatomy of the patient, including one or more surgical navigation markers positioned on the patient, for display on the display device.

U.S. Patent Application 2011/0004259 to Stallings et al. describes a device for positioning a fiducial marker on an anatomical structure. The device includes a fiducial base and a fixation member. The fiducial base comprises a turn and an extension configured to position the fiducial marker within the field of view of a tracking sensor.

U.S. Patent Application 2011/0098553 to Robbins et al. describes automatic registration of a magnetic resonance (MR) image is carried out in an image guidance system by placing MR visible markers at known positions relative to markers visible in a camera tracking system.

U.S. Patent Application 2015/0150641 to Doan et al. describes a position and orientation tracking system having one or more pattern tags, each tag comprising a plurality of contrasting portions. There is a tracker for obtaining image information about the pattern tags, and a database with geometric information describing patterns on the pattern tags.

U.S. Patent Application 2015/0366620 to Cameron et al. describes a guide for use with an access port for port-based surgery. The guide includes a body positionable over a surgical opening and a grip coupled to the body for removably receiving the access port into the surgical opening.

U.S. Patent Application 2017/0281283 to Siegler et al. describes tracking marker support structures that include one or more fiducial reference markers, where the tracking marker support structures are configured to be removably and securely attached to a skeletal region of a patient.

U.S. Patent Application 2018/0200002 to Kostrzewski describes robotic surgical systems with built-in navigation capability for patient position tracking and surgical instrument guidance during a surgical procedure, without the need for a separate navigation system.

U.S. Patent Application 2018/0318035 to McLachlin et al. describes a reference tie that is to be secured around a portion of a spine during a surgical procedure and that is to be tracked by a surgical navigation system.

U.S. Patent Application 2019/0015163 to Abhari et al. describes how navigational information relative to a site of a medical procedure is determined. The navigational information is then mapped to a common coordinate space, to determine the navigational information relative to a field of view of saved and live optical images of the surgical site.

Documents incorporated by reference in the present patent application are to be considered an integral part of the application except that, to the extent that any terms are defined in these incorporated documents in a manner that conflicts with definitions made explicitly or implicitly in the present specification, only the definitions in the present specification should be considered.

### SUMMARY OF THE INVENTION

An embodiment of the present invention provides a medical marking device, including:
a radiotransparent plate having a first plurality of radiopaque elements embedded therein in a first predefined pattern and a second plurality of optical reflectors positioned on a surface in proximity to the plate in a second predefined pattern; and
a sigmoid mounting arm having a first end connected to the radiotransparent plate and a second end containing one or more fastening receptacles.

Typically, the sigmoid mounting arm is radiotransparent.

In a disclosed embodiment the sigmoid mounting arm consists of a first curved section connected to a second curved section by a straight section.

In a further disclosed embodiment the sigmoid mounting arm consists of a first curved section connected directly to a second curved section.

In a yet further disclosed embodiment the first and the second predefined patterns have no axes of symmetry and no planes of symmetry.

There is further provided, according to an embodiment of the present invention, apparatus, including:
a surgical clamp for attachment to a bone of a patient;
a spacer having a first surface and a second surface configured to connect fixedly to the surgical clamp; and
a marking device, which includes:
a radiotransparent plate having a plurality of radiopaque elements embedded therein in a predefined pattern; and
a sigmoid mounting arm having a first end fixedly connected to the radiotransparent plate and a second end, configured to mate with the first surface of the spacer, and containing one or more fastening receptacles configured for removable connection of the arm from the spacer.

In an alternative embodiment there is a fixed distance between the first and spacer surfaces.

In a further alternative embodiment the first surface includes a first plate and the second surface includes a second plate, the apparatus further having an adjustable mechanism connecting the first and the second plate configured to adjust a separation of the plates.

There is further provided, according to an embodiment of the invention, apparatus, including:
a surgical clamp for attachment to a bone of a patient;
clamp adjustment elements configured to effectuate the attachment;
a support structure enclosing the clamp adjustment elements; and
a marking device, which consists of:
a radiotransparent plate having a plurality of radiopaque elements embedded therein in a predefined pattern; and
a sigmoid mounting arm having a first end fixedly connected to the radiotransparent plate and a second end containing one or more fastening receptacles configured for removable connection of the arm from the support structure.

There is further provided, according to an embodiment of the invention, a method, including:
embedding a first plurality of radiopaque elements, arranged in a first predefined pattern, in a radiotransparent plate;
positioning a second plurality of optical reflectors on a surface in proximity to the plate in a second predefined pattern; and
connecting a first end of a sigmoid mounting arm to the radiotransparent plate, the arm having a second end containing one or more fastening receptacles.

There is further provided, according to an embodiment of the invention, a method, including:
attaching a surgical clamp to a bone of a patient;
embedding a first plurality of radiopaque elements, arranged in a first predefined pattern, in a radiotransparent plate and positioning a second plurality of optical reflectors on a surface in proximity to the plate in a second predefined pattern;
connecting a first end of a sigmoid mounting arm to the surgical clamp, the arm having a second end fixedly connected to the radiotransparent plate;
fluoroscopically scanning the radiotransparent plate, the sigmoid mounting arm, and the surgical clamp so as to form a fluoroscopic scan;
optically scanning the second plurality of optical reflectors so as to form a first optical scan;
disconnecting the first end of the sigmoid mounting arm from the surgical clamp and connecting a patient marker, having a third plurality of reflectors in a third predefined pattern, to the surgical clamp;
optically scanning the third plurality of optical reflectors so as to form a second optical scan;
deriving from the first and the second optical scans a correction vector indicative of a difference between a location and an orientation of the radiotransparent plate and a location and an orientation of the patient marker; and
in response to the correction vector and the fluoroscopic scan, determining a spatial transformation between the patient and the surgical clamp so as register a frame of reference of the patient with a frame of reference of the surgical clamp.

Typically, the method includes, in response to registration of the frames of reference, presenting to a professional performing surgery on the patient a stored image of the patient aligned with the patient.

The present disclosure will be more fully understood from the following detailed description of the embodiments thereof, taken together with the drawings, in which:

### BRIEF DESCRIPTION OF THE DRAWINGS

FIG. 1 is a schematic diagram illustrating a medical marking device as it is used during a preparatory stage of a medical procedure, according to an embodiment of the present invention;

FIG. 2 is a schematic view of the device separated from a clamp, according to an embodiment of the present invention;

FIG. 3 is a schematic exploded view of the device, according to an embodiment of the present invention;

FIG. 4 is a flowchart of steps performed in both a preparatory stage and a subsequent stage of a procedure, according to an embodiment of the present invention;

FIG. 5 is a schematic illustration of the subsequent stage of the procedure, according to an embodiment of the present invention; and

FIGS. 6, 7, and 8 are schematic views of a medical marking device, according to alternative embodiments of the present invention.

### DETAILED DESCRIPTION OF EMBODIMENTS

### Overview

An embodiment of the present invention provides a fiducial marker that is used to register different frames of reference that are present in image guided surgery on a patient. The image guided surgery may be performed by a medical professional wearing an augmented reality head-mounted display, and in order for the display to operate correctly, images of the patient presented to the professional should align with the actual patient. The registration provided by the fiducial marker ensures the necessary image alignment.

In a disclosed embodiment the marker is connected to a clamp which has been attached, in a preparatory stage of a procedure performed on a patient, to one or more spinous processes of the patient. To accommodate physical differences between patients, embodiments of the invention provide alternative systems for attaching the fiducial marker to the clamp, the alternative systems including fixed width and variable width spacers connecting the marker to the clamp.

The fiducial marker comprises radiopaque elements arranged in a predetermined pattern, so that a computerized tomography (CT) scanned image of the marker and of the patient enables frames of reference of the patient's anatomy and of the clamp to be registered in the preparatory stage.

In a subsequent stage of the procedure the fiducial marker is replaced by a patient marker. In some embodiments of the invention the fiducial marker also comprises optical reflectors and images of these may be used to accommodate different positions of the patient marker.

The registration derived in the preparatory stage is used in the subsequent stage of the procedure so that images of the patient marker are used to track the patient, and to ensure that images presented to the professional are correctly aligned.

The CT facility typically comprises an intraoperative CT scanner which has a narrow field of view, so that for the registration to be successful, the radiopaque elements of the fiducial marker and the vertebral bodies should be close. Embodiments of the invention achieve this close proximity by having the fiducial marker in the form of a “step,” with one part of the step comprising a plate containing the radiopaque elements, the step-like form of the fiducial marker enabling the plate to be positioned close to the vertebral bodies. A second part of the step attaches to the clamp, and there is a known mechanical offset between the two parts.

In embodiments of the present invention the plate is radiotransparent, and has the plurality of radiopaque elements referred to above embedded therein in the predetermined pattern. To achieve the step-like form described above, a first end of a sigmoid mounting arm is fixedly connected to the radiotransparent plate. In addition, the sigmoid mounting arm has a second end containing one or more fastening receptacles that are configured for removable connection of the arm to the clamp that is attached to the spinous processes.

In a disclosed embodiment the radiotransparent plate, the sigmoid mounting arm, and the surgical clamp are scanned fluoroscopically so as to form a fluoroscopic scan. In response to the fluoroscopic scan and the predetermined mechanical offset, a spatial transformation between the patient and the surgical clamp is determined so as to register a frame of reference of the patient with a frame of reference of the surgical clamp.

Typically, in response to registration of the frames of reference, a professional performing surgery on the patient is presented with a stored image of the patient aligned with the patient.

While the description herein refers to a fiducial marker or a patient marker being connected to a clamp that is attached to one or more spinous processes of vertebrae of a patient, it will be understood that the scope of the invention comprises any marker retaining structure, not just a clamp, that is rigidly attached to any bone of the patient. For example, the retaining structure may comprise an iliac pin that is rigidly inserted into an ilium of the patient. As is the case for the clamp, a fiducial marker or a patient marker may be connected to the retaining structure.

The different frames of reference referred to above comprise a patient frame of reference, a clamp frame of reference, a fiducial marker frame of reference, and a patient marker frame of reference. As is described herein, during the course of a procedure on the patient, the different frames of reference are registered with each other, so that during the image guided surgery images presented to the professional wearing the head mounted display align with the patient.

The registration between any two of the different frames of reference may be by imaging the two associated different elements in a single image, and calculating the registration between the frames of reference from the imaged elements in the single image. For example, the fiducial marker frame of reference may be registered with the patient frame of reference by acquiring then analyzing a fluoroscopic image of the fiducial marker and of a bone of the patient, after the fiducial marker has been rigidly attached to the bone. Alternatively, in the case that the clamp is visible in the fluoroscopic image, both the fiducial marker frame of reference and the clamp frame of reference may be registered with the patient frame of reference by analysis of the image.

Alternatively or additionally, the registration between any two frames of reference may be accomplished if the physical dimensions of the two associated different elements, and how the elements are physically connected together, are known or may be determined. For example, as is described in more detail below, the dimensions of the patient marker and the clamp, and how the patient marker is connected to the clamp, are known. From the known dimensions and method of connection, the frames of reference of the patient marker may be registered with the clamp.

Since the clamp is rigidly attached to the patient, the registrations between the frames of reference of the patient marker and the clamp, and between the frames of reference of the clamp and the patient are transitive relations, Consequently the two registrations may be combined, as is described below, to form a direct registration between the frames of reference of the patient marker and the patient.

In cases where an image of the clamp is not available, it will be understood that such a direct registration may be formed, without using an image of the clamp as an intermediary, if the dimensions of the patient marker and the clamp, and their physical and method of connection, are known, and if the dimensions and physical method of connection of the clamp to the patient are known. In this case dimensions directly relating the patient marker to the patient are known, and these may be used to calculate the registration between the two entities.

### System Description

In the following, all directional references (e.g., upper, lower, upward, downward, left, right, top, bottom, above, below, vertical, and horizontal) are only used for identification purposes to aid the reader's understanding of the present invention, and do not create limitations, particularly as to the position, orientation, or use of embodiments of the invention.

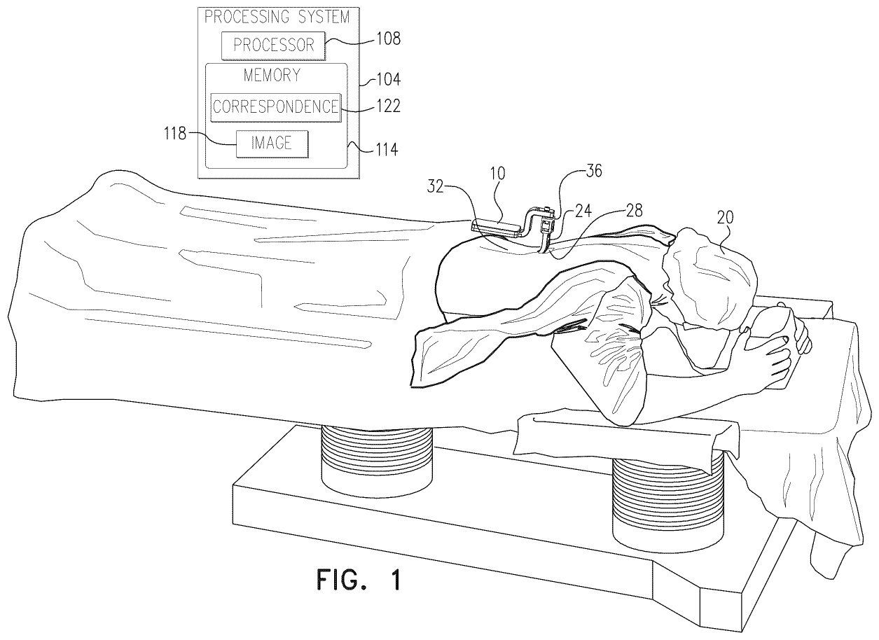

Reference is now made to FIG. 1, which is a schematic diagram illustrating a medical marking device 10, as it is used during a preparatory stage of a medical procedure on a patient 20, according to an embodiment of the present invention. The procedure referred to herein is assumed to comprise a preparatory stage and a subsequent stage, and, as is described in more detail below, device 10 may be used during the preparatory stage. Device 10 is also herein termed a fiducial marker 10. As is also described below, a medical professional uses an augmented reality system during the subsequent stage of the procedure.

The augmented reality system projects virtual images of elements of patient 20 for viewing by the medical professional. The image projection is performed simultaneously with the professional viewing the actual patient, so that the projected images should align with the patient. In order for the projected images in the augmented reality system to align with the patient, frames of reference of the patient and of a clamp attached to a bone of the patient are registered during the preparatory stage, using marking device 10. In the subsequent stage, where the augmented reality system is used, the registration of the clamp computed in the preparatory phase is used to correctly align the images produced by the system.

In the description herein the procedure is assumed to comprise an operation on a spine of patient 20, and in order to perform the registration referred to above, prior to the operation, and in the preparatory stage of the procedure, the medical professional inserts a surgical bone clamp 24 into patient 20. A site of insertion 28 of the clamp is close to, but separate from, a site 32 of the patient's spine to be operated on during the subsequent stage of the procedure. The professional clamps clamp 24 to a section of the spine of the patient, typically to one or more spinous processes of the patient, and the clamp has a support structure 36 to which marker 10 is fastened. A clamp similar to clamp 24 is described in U.S. patent application Ser. No. 16/120,480 which is incorporated herein by reference.

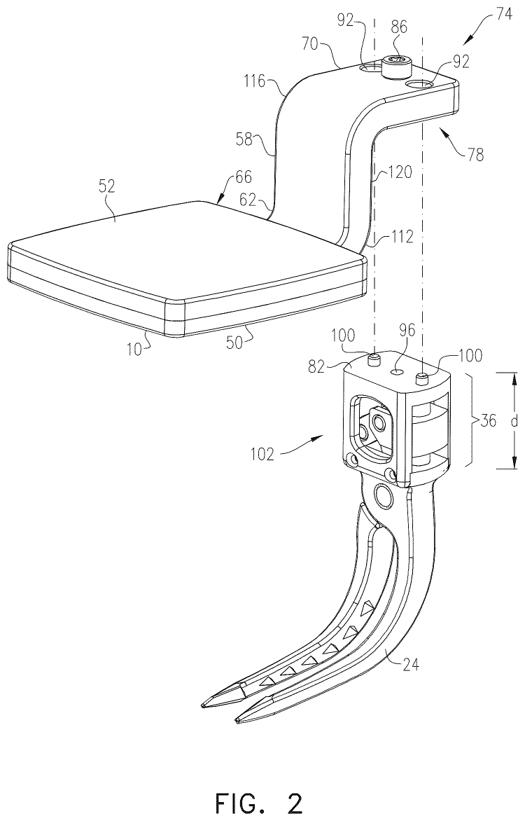

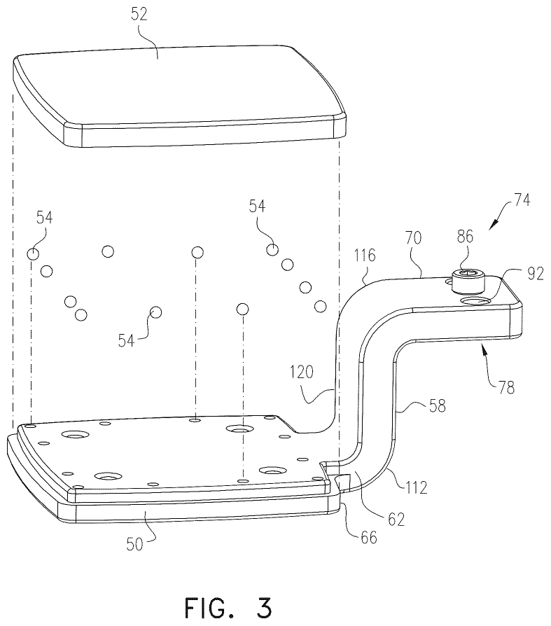

FIG. 2 is a schematic view of device 10 separated from clamp 24, and FIG. 3 is a schematic exploded view of device 10, according to an embodiment of the present invention. Device 10 comprises a radiotransparent plate 50, typically formed of a biocompatible plastic such as a polyimide. A plurality of substantially similar radiopaque elements 54, herein assumed to comprise spheres, are embedded within plate 50. Elements 54 are embedded within plate 50 in a predefined pattern, which is herein assumed to comprise points on sides of a rectangle. However, any other convenient predefined pattern may be used. In some embodiments the predefined pattern has no axis or plane of symmetry. Typically plate 50 is protected by a cover 52.

A radiotransparent sigmoid mounting arm 58, typically formed from the same biocompatible plastic as plate 50, is connected by a first end 62 of the arm to an edge 66 of the plate. A second end 70 of the arm contains one or more fastening receptacles 74 that enable the arm to be securely and fixedly fastened to support structure 36 in a predefined orientation. In embodiments of the invention, support structure 36 encloses adjustment elements 102 for clamp 24, the adjustment elements effectuating attachment of jaws of the clamp to a bone of patient 20. In an embodiment of the invention the support structure has differing depths d.

Second end 70 has a lower plane surface 78 which connects with an upper surface 82 of support structure 36. In a disclosed embodiment receptacles 74 comprise a captive screw 86 and holes 92, the screw and the holes mating respectively with a threaded hole 96 and studs 100 in upper surface 82.

As is apparent from the figures, the length of arm 58 determines the separation of plate 50 from lower surface 78 of the arm second end. Embodiments of the present invention comprise sets of devices 10, each member of the set being generally as described herein, but having a different known length of arm 58 from the other set members. Arm 58 is formed from two curved sections 112, 116, separated by a straight section 120, and the different lengths of the arm are formed by varying a length of straight section 120. In embodiments of the present invention the length of straight section 120 varies from 0 (zero) to up to approximately 7 cm, although values greater than 7 cm are possible. It will be understood that when the length of section 120 is 0 the two curved sections are connected together with no intervening straight section.

Each member of the set, comprising a respective plate 50 connected to a respective sigmoid mounting arm 58 in a step-like arrangement, may be formed as a single piece, typically by injection molding. It will be understood that each member of the set has known dimensions, so that there is a known mechanical offset between plate 50 and the second end of the arm, including the lower surface 78 of the arm second end.

Typically, for each member of a set of devices 10, the predefined pattern of radiopaque elements 54 is configured to have a one-to-one correspondence with the known mechanical offset. In this case, identification of the pattern provides a unique and unambiguous value for the mechanical offset, and providing the correspondence enables embodiments of the invention to support substantially any offset. As is described below, the correspondence may be stored in a computer memory, and the memory may be accessed so that the unique value of the mechanical offset for a given set member may be determined from the element pattern of the member.

In a disclosed embodiment plate 50 and lower surface 78 of device 10 are substantially parallel, and straight section 120 is perpendicular to the plate and to the lower surface. As stated above, the length of straight section 120 may be varied so as to provide sets of devices 10. Typically, a member of the set is selected so that cover 52 is below surface 82 of support structure 36, and so that plate 50 is as close to the skin of patient 20 as possible.

As stated above radiopaque elements are embedded within plate 50 in a predefined pattern, and the pattern may be in a one-one correspondence with the different mechanical offsets provided by the different lengths of straight section 120. For example, if the pattern is a rectangle, then the sides of the rectangle may be configured so that a different ratio of the lengths of the sides corresponds to a respective straight section length. Alternatively, the number of elements in a rectangle side may be varied, and the number configured to correspond to a length of section 120. As another example the shape of the pattern may be varied, and a different shape may be configured to correspond to a respective straight section length. Shapes may comprise a triangle, a quadrilateral, a pentagon, etc., but any convenient shape may be used.

In addition to the sets of devices 10 described above, embodiments of the present invention also comprise sets of clamps 24 with differing depths d of support structure 36. It will be understood that for any given arm length 58, the depth d of support structure 36 determines the separation of plate 50 from a given clamp selected from the sets of clamps 24.

Returning to FIG. 1, device 10 is typically selected from the set of devices 10, and clamp 24 is typically selected from the set of clamps 24 so that when the device is attached to upper surface 82 of support structure 36, plate 50 of the device is as close as possible to site 32, i.e., to the site of the operation to be performed in the procedure subsequent stage. Once a selected device 10 has been attached to surface 82, a computerized tomography (CT) scan of the device and of the patient's spine is performed. The scan may be performed by inserting patient 20 into a CT scanning facility, typically an intra-operative CT scanner. The insertion may be implemented by bringing the CT scanning facility to patient 20, or by transporting the patient to the facility.

A processing system 104, comprising a computer processor 108 coupled to a memory 114, receives the scan of device 10 and the patient's spine, and stores the scan as an image 118 in the memory. The one-to-one correspondence referred to above may also be stored in memory 114 as a correspondence 122. The processing system is configured to analyze the stored image so as to identify the pattern formed by radiopaque elements 54, and from the identified pattern to register a frame of reference of device 10, and thus of attached clamp 24, with a frame of reference of the patient's anatomy.

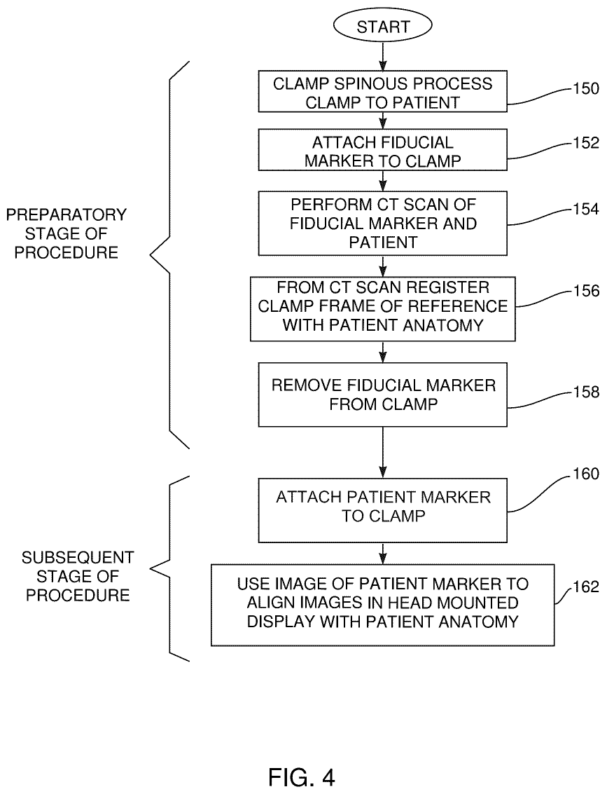

FIG. 4 is a flowchart of steps performed in both the preparatory stage and the subsequent stage of the procedure, according to an embodiment of the present invention. In an initial step 150 and in an attachment step 152 of the preparatory stage, clamp 24 is inserted into patient 20 and clamped, using adjustment elements 102, to one or more spinous processes of the patient, and fiducial marker 10 is attached to support structure 36 of the clamp, as is described above with reference to FIG. 1. Steps 150 and 152 are performed by a user of the invention, typically professional 180 (referred to below).

In a scan step 154 a CT scan of marker 10, attached as described above, is performed, and image 118 of the scan is stored in memory 114. The CT scan may be accessed and stored by the processor of processing system 104. In an analysis step 156 the processing system analyzes the stored image, and from the analysis registers a frame of reference of marker 10, and thus of attached clamp 24, with a frame of reference of the patient's anatomy.

It will be appreciated that the registration of step 156 uses the known mechanical offset of plate 50 with lower surface 78 to provide a location and orientation of upper surface 82 of the clamp with respect to the frame of reference of the patient's anatomy. In the cases where the one-to-one correspondence referred to above is stored as correspondence 122 in memory 114, the processing system may determine the mechanical offset, in analysis step 156, from the pattern identified by the analysis, using the stored correspondence.

Analysis step 156 also determines a spatial transformation between the patient and the surgical clamp, and this is used, together with the mechanical offset, to calculate the registration between the two frames of reference.

In a concluding step 158 of the preparatory stage, fiducial marker 10 is removed from clamp 24, leaving upper surface 82 exposed. The user of the invention performs concluding step 158.

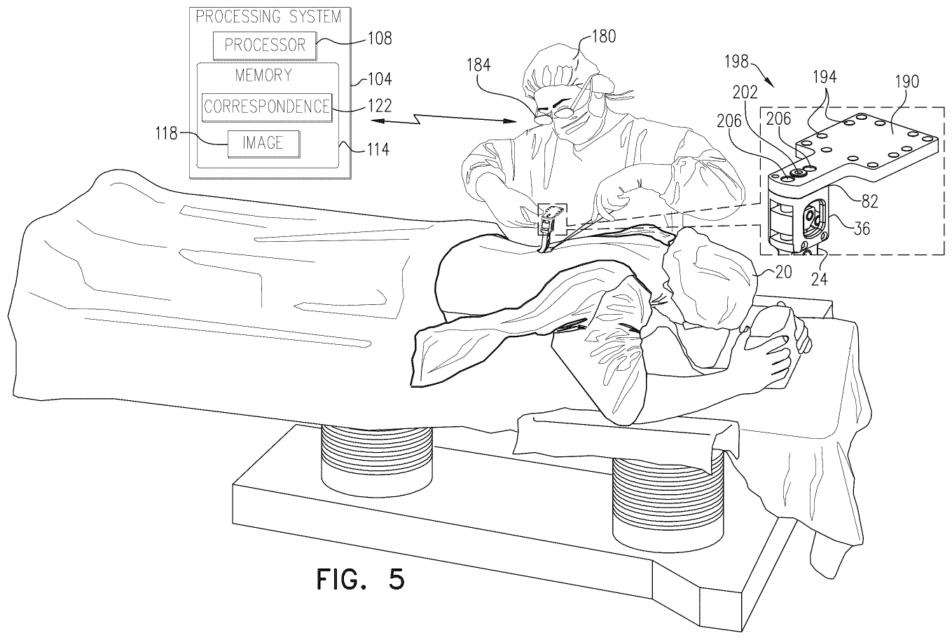

FIG. 5 is a schematic illustration of the subsequent stage of the procedure, according to an embodiment of the present invention. In the subsequent stage a patient marker attachment step 160 and an alignment step 162 are performed, and these steps are described further below. In the subsequent stage, a medical professional 180 operates on the patient. Professional 180 wears an augmented reality head-mounted display (HMD) 184, which is configured to present stored images that are aligned with the patient, to the professional. In order to operate, HMD 184 is coupled to processor 108 of system 104 and the stored images may be stored in memory 114. Alternatively, HMD 184 has its own dedicated processor which performs similar functions to those performed by processor 108. A head-mounted display similar to HMD 184 is described in U.S. Pat. No. 9,928,629, which is incorporated herein by reference.

To perform the alignment for HMD 184, in attachment step 160 a patient marker 190 with known, preset, dimensions, is attached to upper surface 82 of support structure 36 of clamp 24. Patient marker 190 may be attached in a selected pre-determined orientation. Marker 190 comprises fastening receptacles 198, substantially similar to receptacles 74, so that in the disclosed embodiment referred to above receptacles 198 comprise a captive screw 202 and holes 206, the screw and the holes mating respectively with threaded hole 96 and studs 100 in upper surface 82 of clamp 24. A patient marker similar to marker 190 is described in PCT Patent Application PCT/IB2019/053524, which is incorporated herein by reference. Attachment step 160 is performed by professional 180. As is described in PCT Patent Application PCT/IB2019/053524, a patient marker such as marker 190 may be configured to be attached in a number of discrete pre-determined orientations to upper surface 82. Thus, all such pre-determined orientations for patient marker 190 are assumed to be comprised within the scope of the present invention.

Marker 190 comprises optical reflectors 194 incorporated into the surface of the patient marker, and the reflectors are arranged in a predetermined pattern so that an image of the reflectors can be analyzed so as to provide an unambiguous measure of the location and of the orientation of the marker.

In alignment step 162 the HMD projects visible or invisible light to patient marker 190, and acquires images of reflectors 194 of the marker. From the acquired images, the HMD processor determines the location and orientation of the patient marker. Since the patient marker has known dimensions, and is attached to upper surface 82, the processor applies the registration found in step 156 (between the frames of reference of fiducial marker 10 and the patient's anatomy) to ensure that the images projected by the HMD align with the anatomy of patient 20. Alignment step 162 is performed by processing system 104.

As is stated above, a user performs steps 150, 152, 158, and 160, and processor 108 of processing system 104 performs the access and storage portions of step 154, as well as steps 156, and 162. Thus, in the user steps described above, professional 180 inserts clamp 24 into patient 20, and attaches the clamp to the spinous processes of the patient. The professional then attaches fiducial marker 10 to the clamp. In some embodiments, the professional selects the fiducial marker from a set of different fiducial markers so that plate 50 is as close to the skin of patient 20 as possible. Thus, the professional chooses the fiducial marker with a length of arm 58, that achieves this goal. In some embodiments the clamp has different lengths, and there may be a spacer (described in more detail below) connecting the marker to the clamp. In this case the operator may select one or a combination of the clamp, the spacer (if used) and/or the fiducial marker to achieve the goal. At the conclusion of the preparatory stage of the procedure, and at the beginning of the subsequent stage, the professional detaches the fiducial marker and any spacer that has been used, and attaches patient marker 190 to clamp 24.

In performing the portions of step 154, and steps 156, and 162, processor 108 accesses and stores an acquired CT scan of the attached fiducial marker, and stores an image of the scan in memory 114. The processor then analyzes the image so as to register a frame of reference of the fiducial marker with a frame of reference of the patient's anatomy. Once the fiducial marker has been detached from the clamp, and the patient marker has been attached, the processor acquires or accesses an image of the attached patient marker, and analyzes the image to determine the location and orientation of the patient marker with respect to the operator. Using the known spatial dimensions of the patient marker and its attachment in a known, determined, orientation to the clamp, the processor uses the registration referred to above, and the acquired image of the patient marker, to ensure that the projected HMD images align with the anatomy of patient 20 as seen from the point of view of the user.

In an alternative embodiment, rather than calculating the registration between the frames of reference of the patient and the fiducial marker, as is described above for scan step 154 and analysis step 156, in step 156 the processing system uses the scan of step 154, wherein the CT scan scans the fiducial marker and the patient's anatomy, to find physical dimensions relating upper surface 82 of the clamp to the attached spinous processes of the patient.

In the alternative embodiment, in alignment step 162, the HMD processor uses the images of reflectors 194 to unambiguously determine the location and orientation of patient marker 190, which is attached to upper surface 82 of the clamp. Alternatively, the location and orientation of the patient marker may be determined unambiguously by any convenient method known in the art.

In the alternative embodiment HMD processor uses the known dimensions of the patient marker, and its unambiguously determined location and orientation with respect to the clamp, together with the physical dimensions relating the clamp to the patient's spinous processes, to calculate a spatial relationship between the patient marker and the spinous processes. It will be understood that the spatial relationship enables the HMD processor to form a direct registration between the frames of reference of the patient marker and the spinous processes, i.e., the patient's anatomy, so that the processor does not need to calculate the registrations between the frames of reference of the patient marker and the clamp, and between the frames of reference of the clamp and the patient's anatomy.

The description above provides details of how fiducial marker 10 and patient marker 190 may be attached, via clamp 24, as close as possible to site 32, i.e., to the site of the operation performed on patient 20. The following description provides further details of embodiments of the invention which may be used, alternatively or additionally, so that the patient marker and the fiducial marker are as close as possible to the site of the operation.

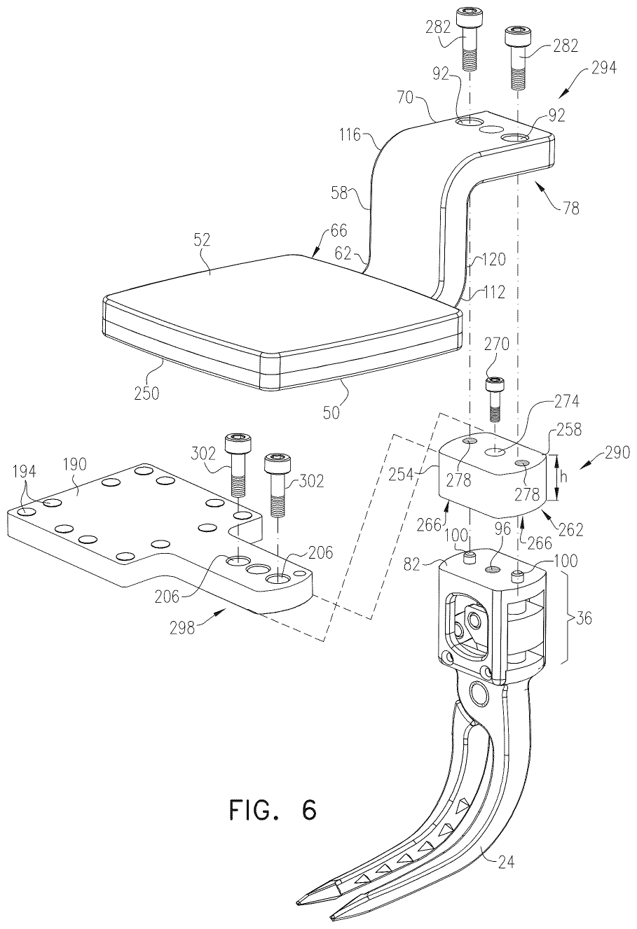

FIG. 6 is a schematic view of a medical marking device 250 separated from clamp 24, according to an alternative embodiment of the present invention. Apart from the differences described below, the operation of device 250 is generally similar to that of device 10 (FIGS. 1-5), and elements indicated by the same reference numerals in both devices 250 and 10 are generally similar in construction and in operation. Device 250 is herein also termed fiducial marker 250.

In contrast to device 10, wherein second end 70 of the sigmoid arm is configured to connect directly to upper surface 82 of support structure 36, in device 250 there is an intervening spacer 254 between the sigmoid arm second end and surface 82. Spacer 254 is formed to have a pair of opposing parallel surfaces, an upper spacer surface 258 and a lower spacer surface 262, separated by a preset fixed distance h. When assembled, lower surface 262 fixedly mates with upper surface 82 of support structure 36, and lower surface 78 of the sigmoid arm removably mates with upper surface 258 of spacer 254.

So that lower spacer surface 262 can fixedly mate with support structure surface 82, the lower spacer surface has two blind holes 266 inlet into the lower spacer surface that align with and fit to studs 100. In addition, spacer 254 comprises a captive screw 270, residing in a hole 274 of the spacer. The captive screw is configured so that when it is screwed into threaded hole 96 it fastens the spacer fixedly to support structure 36, while the head of screw 270 is within hole 274 and below upper surface 258 of the spacer. Captive screw 270 and blind holes 266 act as spacer lower surface fastening receptacles 290.

Once spacer 254 is fixed to support structure 36, sigmoid arm lower surface 78 may be removably mated with the upper surface of the spacer. The spacer upper surface comprises two threaded holes 278 which align with holes 92. To removably mate the sigmoid arm lower surface with the spacer, a pair of retaining screws 282 pass through holes 92, and are screwed into threaded holes 278. Screws 282 and threaded holes 278 act as spacer upper surface fastening receptacles 294.

Spacer 254 is typically part of a set of such spacers. In one embodiment spacers of the set have values of h in a range of 1 cm-5 cm, but in other embodiments the set has different ranges of h. Spacer 254 is used to raise plate 50 above clamp 24 while keeping the plate as close as possible to the patient's spine, and the value of h of a selected spacer may be chosen according to patient characteristics. It will be understood that having a set of spacers 254 with different values of h replaces, or is in addition to, the requirement of different lengths of arm 58 referred to above for a set of devices 10.

During a procedure where marker 250 is used, after it has been attached, as described above, to the upper surface of spacer 254, it may be scanned fluoroscopically, and may then be removed from the spacer upper surface. After removal, as illustrated in FIG. 6, patient marker 190 may then be attached to the spacer upper surface. The patient marker attachment is implemented by screws 302, substantially similar to screws 282, penetrating holes 206 and being screwed into threaded holes 278. Screws 302 and threaded holes 278 act as fastening receptacles 298 for the patient marker, receptacles 298 being substantially similar to receptacles 294.

Referring back to the flowchart of FIG. 4, when marker 250 is used in place of marker 10, the actions described above for each of the steps of the flowchart are substantially similar, except as follows.

In step 152, spacer 254 is first fixedly attached to support structure 36, and then fiducial marker 250 is removably attached to the spacer, as described above.

In step 158 fiducial marker 250 is removed from the spacer, and in step 160 patient marker 190 is attached to the spacer, as described above.

It will be understood that because fiducial marker 250 and patient marker 190 are attached to the same location, the upper surface of the spacer, the registration found in step 156 can be used in alignment step 162.

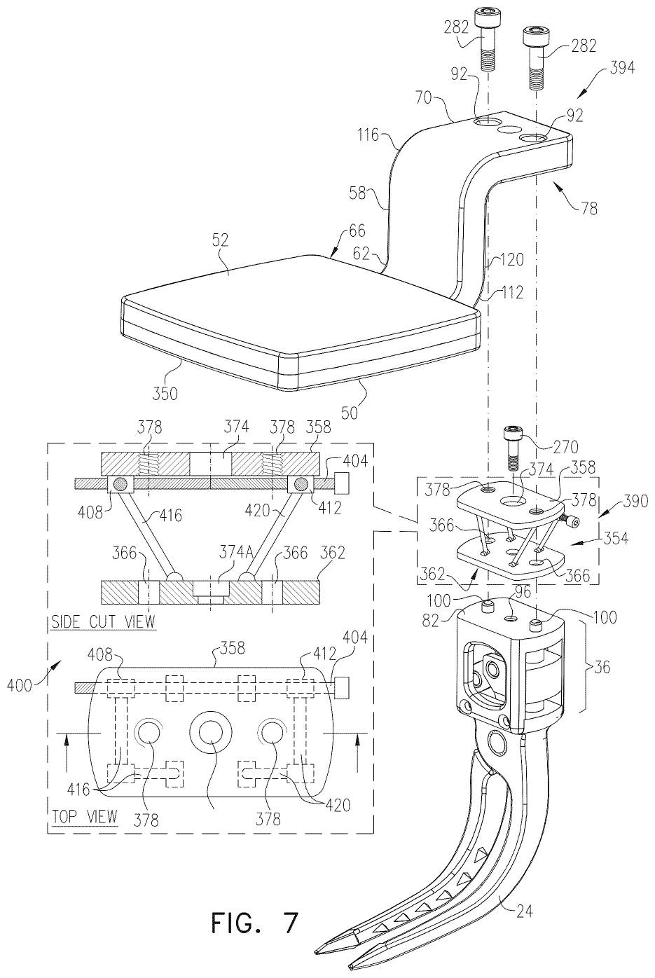

FIG. 7 is a schematic view of a medical marking device 350 separated from clamp 24, according to an alternative embodiment of the present invention. Apart from the differences described below, the operation of device 350 is generally similar to that of device 250 (FIGS. 1-6), and elements indicated by the same reference numerals in both devices 250 and 350 are generally similar in construction and in operation. Device 350 is herein also termed fiducial marker 350.

In contrast to device 250, which uses spacer 254 with a preset fixed distance between the upper and lower surfaces of the spacer, device 350 uses a spacer 354, herein also termed adjustable spacer 354, which has adjustable distances between an upper adjustable spacer plate 358 and a lower adjustable spacer plate 362. Elements in the two plates of adjustable spacer 354 correspond to respective elements in the two surfaces of fixed spacer 254. Thus, in upper plate 358 threaded holes 378 and a hole 374 are substantially similar to holes 278 and 274, so that hole 374 permits passage of captive screw 270. Screws 282 and threaded holes 378 act as upper adjustable spacer plate fastening receptacles 394.

In lower plate 362 holes 366 are substantially similar in diameter and position to blind holes 266, although holes 366 are not necessarily blind. Lower plate 362 also comprises a hole 374A, which aligns with hole 374 and which is sized to retain screw 270. Captive screw 270 and holes 366 act as lower adjustable spacer plate fastening receptacles 390.

Adjustable spacer 354 comprises an adjustable mechanism 400, which connects the lower and upper plates of adjustable spacer 354. Mechanism 400 maintains the upper plates substantially parallel to each other, while operation of the mechanism adjusts the separation of the plates.

In one embodiment, illustrated in FIG. 7, mechanism 400 comprises a rod 404 having clockwise and counterclockwise threads, and the threads are configured to mate with respective nuts 308, 412 slidingly attached to upper plate 358. Mechanism 400 further comprises two levers 416, 420, having their fulcra coupled to lower plate 362, and their endpoints attached to the respective nuts. Rotation of rod 404 of mechanism 400 increases or decrease the nut separation, and correspondingly decreases or increases the separation between the upper and lower plates of adjustable spacer 354.

Other mechanisms for connecting the upper and lower plates of spacer 354, that maintain the plates substantially parallel to each other while providing adjustable separation of the plates, will be apparent to those having ordinary skill in the art. All such mechanisms are assumed to be comprised within the scope of the present invention.

Referring back to the flowchart of FIG. 4, when marker 350 is used in place of marker 10, the actions described above for each of the steps of the flowchart are substantially similar, except as follows.

In step 152, lower plate 362 is first fixedly attached to support structure 36, and then fiducial marker 350 is removably attached to upper plate 358, as described above.

In step 158 fiducial marker 350 is removed from the spacer upper plate 358, and in step 160 patient marker 190 is attached to the spacer upper plate, as described above.

It will be understood that because fiducial marker 350 and patient marker 190 are attached to the same location, the upper surface of the spacer upper plate, the registration found in step 156 can be used in alignment step 162.

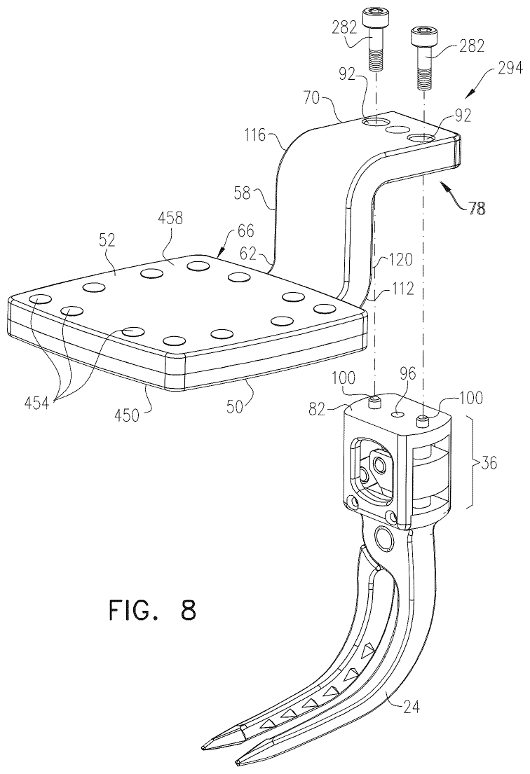

FIG. 8 is a schematic view of a medical marking device 450 separated from clamp 24, according to an alternative embodiment of the present invention. Apart from the differences described below, the operation of device 450 is generally similar to that of device 250 (FIGS. 1-6), and elements indicated by the same reference numerals in both devices 250 and 450 are generally similar in construction and in operation. Device 450 is herein also termed fiducial marker 450.

In contrast to device 250, a plurality of optical reflectors 454 are positioned on a surface 458 of cover 52 of plate 50. In one embodiment reflectors 454 are arranged on the surface in a predetermined pattern, typically with no rotational axis of symmetry (other than a trivial axis of symmetry for rotating by) 360° and no mirror plane of symmetry. The predetermined pattern is configured so that an image of the reflectors can be analyzed so as to provide an unambiguous measure of the location and of the orientation of plate 50. The imaging of reflectors 454, and the analysis of the image, is substantially as described above for reflectors 194 of patient marker 190.

Device 450 is typically used with spacer 254 and/or spacer 354, described above with reference to FIGS. 6 and 7. When used device 450 is used with one of these spacers, patient marker 190 may be positioned with respect to the spacer as described above. However, because of the presence of reflectors 454, patient marker 190 may instead be positioned in any other convenient location, as is explained below with reference to changes in the flowchart of FIG. 4.

For clarity, in the following description of changes to the flowchart, device 450 is assumed to be used with spacer 254. Those having ordinary skill in the art will be able to adapt the description, mutatis mutandis, if device 450 is used with spacer 354 or with any other spacer effectively separating plate 50 from support structure 36.

In step 152 of the flowchart, spacer 254 is first attached to support structure 36 using screw 270, and then fiducial marker 450 is attached to the spacer using screws 282, as described above.

Steps 154 and 156 are performed as described above with reference to FIG. 4, so as to register a frame of reference of marker 450, and thus of attached clamp 24, with a frame of reference of the patient's anatomy.

In step 158, prior to removal of fiducial marker 450 from the spacer, reflectors 454 of the marker are optically scanned and are imaged using HMD 184. The processor associated with HMD 184 analyzes the image to find the location and orientation of the reflectors, and thus of plate 50, in the frame of reference of the HMD. Marker 450 may then be removed from spacer 254, and the spacer may be removed from support structure 36.

Steps 160 and 162 are implemented generally as described above, so that in step 160 patient marker 190 is attached to upper surface 82 of support structure 36.

In step 162 patient marker 190 is optically scanned and is tracked, and the processor of the HMD is able to use the location and orientation of plate 50 found in step 158 to generate a correction vector, between the positions of the plate and the positions of the patient marker. The correction vector corrects for the fact that the fiducial marker and the patient marker are attached to different locations (i.e., the spacer and the support structure). In step 162 the HMD processor applies this correction vector to ensure that the images projected by the HMD align with the anatomy of patient 20.

The description above is for a particular case where patient marker 190 and fiducial marker 450 are attached to different elements related to clamp 24. Those having ordinary skill in the art will be able to adapt the description, mutatis mutandis, for other cases of different attachments, and all such cases are assumed to be comprised with the scope of the present invention.

The scans in steps 158 and 162 are separate scans, so that typically the HMD is in different locations for the scans. In step 158, because of the known dimensions of fiducial marker 450 and spacer 254, the processor associated with the HMD is able to find a local fiducial marker vector relating the location of plate 50 to lower surface 78, and thus to upper surface 82 of support structure 36. It will be appreciated that this method of finding a local fiducial marker applies to all fiducial markers, with or without a spacer, of the present invention,

In scan 162, patient marker 190 is scanned. The dimensions of the patient marker are known, and reflectors 194 of the patient marker are arranged so that, as stated above, an image of the reflectors is able to provide an unambiguous spatial relation between the operator and the patient marker. The HMD processor analyzes the scanned image of the patient marker (to determine the spatial relation between the operator and the patient marker), and from its determined location and orientation with respect to the patient (or support structure 36) finds a local patient marker vector relating the location of the patient marker to support structure 36, e.g., upper surface 82.

It will be understood that the correction vector between the positions of plate 50 and of patient marker 190 may correspond to the sum of the local fiducial marker vector and the local patient marker vector.

By using the image of the patient marker reflectors to find the location and orientation of the patient marker, it will be understood that even in the case of different possible orientations of the patient marker, other than that illustrated in the figures, the HMD processor is able to find the correction vector. Alternatively or additionally, the processor may be configured to use any other method known in the art for finding the orientation of the patient marker. For example, PCT Patent Application PCT/IB2019/053524, referred to above, describes how the orientation of a patient marker may be determined,

The description above has assumed that during a procedure, device 450 is removed, and is replaced by patient marker 190. However, because of reflectors 454, it will be appreciated that imaging of the reflectors enables device 450 itself to be used as a patient marker. Consequently, in some embodiments of the present invention, device 450 is not removed, and acts both as a fiducial marker and as a patient marker.

Returning to the flowchart of FIG. 4, and applying it also to the embodiments described above with reference to Figs, 6, 7, and 8, a user performs steps 150, 152, 158, and 160, and processor 108 of processing system 104 performs the portions of step 154, and steps 156 and 162. Thus, in the user steps described above, professional 180 inserts clamp 24 into patient 20, and attaches the clamp to the spinous processes of the patient. The professional then attaches fiducial marker 10, 250, 350, or 450 to the clamp. In the cases of markers 250, 350, and 450 the markers are attached using spacer 254 and/or adjustable spacer 354. In selecting which marker to be used, and, where appropriate, which spacer is used, the professional chooses the length of the arm of the marker and the size of the spacer so that plate 50 is as close to the skin of patient 20 as possible. Thus, the professional chooses the fiducial marker with a length of arm 58, and, when spacer 254 is used, the spacer having a value of distance h, that achieves this goal. At the conclusion of the preparatory stage of the procedure, and at the beginning of the subsequent stage, the professional detaches the fiducial marker and any spacer that has been used, and attaches patient marker 190 to clamp 24.

Thus, for the embodiment described above with reference to FIG. 8, as well as the for the other embodiments described herein, in performing the portion of step 154, and steps 156 and 162, processor 108 accesses a CT scan of the attached fiducial marker, and stores an image of the scan in memory 114. The processor then analyzes the image so as to register a frame of reference of the fiducial marker with a frame of reference of the patient's anatomy. Once the fiducial marker has been detached from the clamp, and a patient marker having a known orientation when connected to the clamp, has been attached, the processor accesses or acquires an image of the attached patient marker, and analyzes the image to determine the location and orientation of the operator with respect to the patient marker. Using the known spatial dimensions of the patient marker and its known orientation when connected to the clamp, the processor uses the registration referred to above, and the acquired image of the patient marker, to ensure that the projected HMD images align with the anatomy of patient 20 as seen from the point of view of the user. In addition, as is described above, processor 108 may also use the patient marker image to form a registration between a patient marker frame of reference and a frame of reference of the patient's anatomy.

It will be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. An apparatus for image guided surgery, the apparatus comprising:
a retaining structure for attachment to a bone of a patient; and
a fiducial marker, the fiducial marker comprising:
a radiotransparent plate comprising a plurality of radiopaque elements embedded therein in a predefined pattern; and
a sigmoid mounting arm having a first end fixedly connected to the radiotransparent plate, a second end containing one or more fastening receptacles configured for removable connection of the sigmoid mounting arm to the retaining structure, and a straight section orthogonal to the radiotransparent plate and fixedly connecting the first end to the second end.

2. The apparatus according to claim 1, wherein the retaining structure comprises a clamp, and the bone comprises at least one spinous process of the patient, and wherein the clamp is configured to be clamped to the at least one spinous process.

3. The apparatus according to claim 1, wherein the retaining structure comprises an iliac pin, and the bone comprises an ilium of the patient, and wherein the iliac pin is configured to be rigidly inserted into the ilium.

4. The apparatus according to claim 1, further comprising one or more additional sigmoid mounting arms, each of the one or more additional sigmoid mounting arms having the straight section with a different respective length.

5. The apparatus according to claim 1, further comprising one or more additional fiducial markers, each of the one or more additional fiducial markers having a radiotransparent plate with a different respective predefined pattern for the embedded radiopaque elements and a sigmoid mounting arm having the straight section with a different respective length.

6. The apparatus according to claim 5, wherein each predefined pattern is formed as a group of rectangles, each of the group having a different respective ratio of sides.

7. The apparatus according to claim 5, wherein each predefined pattern is formed as a group of polygons, each of the group comprising a different polygon.

8. The apparatus according to claim 1, wherein on connection of the sigmoid mounting arm to the retaining structure, the radiotransparent plate is below a region of connection of the sigmoid mounting arm to the retaining structure.

9.-18. (canceled)

19. The apparatus according to claim 1, wherein the retaining structure comprises one or more studs positioned to fit within the one or more fastening receptacles of the second end of the sigmoid mounting arm.

20. The apparatus according to claim 19, wherein the one or more studs comprises two studs, and the one or more fastening receptacles comprises two fastening receptacles.

21. The apparatus according to claim 1, wherein the second end of the sigmoid mounting arm comprises a lower plane surface that is positioned above an upper surface of the radiotransparent plate.

22. The apparatus according to claim 1, wherein radiotransparent plate comprises a cover at an upper side of the radiotransparent plate, and wherein the second end of the sigmoid mounting arm is positioned above the cover of the radiotransparent plate.

23. The apparatus according to claim 1, wherein the plurality of radiopaque elements comprise a plurality of radiopaque spheres.

24. The apparatus according to claim 1, wherein the radiotransparent plate comprises a biocompatible plastic.

25. The apparatus according to claim 24, wherein the sigmoid mounting arm comprises the same biocompatible plastic as the radiotransparent plate.

26. The apparatus according to claim 1, wherein the sigmoid mounting arm is radiotransparent.

27. An apparatus for image guided surgery, the apparatus comprising:
a retaining structure for attachment to a bone of a patient; and
a fiducial marker, the fiducial marker comprising:
a radiotransparent plate comprising a plurality of radiopaque elements embedded therein in a predefined pattern; and
a sigmoid mounting arm having a first end fixedly connected to the radiotransparent plate and a second end containing one or more fastening receptacles configured for removable connection of the sigmoid mounting arm to the retaining structure,
wherein the second end of the sigmoid mounting arm comprises a lower plane surface that is parallel to an upper surface of the radiotransparent plate and that is positioned above the upper surface of the radiotransparent plate.

28. The apparatus according to claim 27, wherein the sigmoid mounting arm further comprises a straight section between the first end and the second end, the straight section being orthogonal to the upper surface of the radiotransparent plate.

29. The apparatus according to claim 27, wherein the retaining structure comprises a clamp.

30. The apparatus according to claim 27, wherein the retaining structure comprises a pin.

31. The apparatus according to claim 27, wherein the sigmoid mounting arm is radiotransparent.
